(19) United States
(12) Patent Application Publication
(10) Pub. No.: US 2014/0343143 A1
(43) Pub. Date: Nov. 20, 2014

(54) Use of highly concentrated compositions of selected n-3 fatty acids for the treatment of central nervous system disturbances

(71) Applicant: Tiberio Bruzzese, Milano (IT)

(72) Inventor: Tiberio Bruzzese, Milano (IT)

(21) Appl. No.: 14/451,606

(22) Filed: Aug. 5, 2014

Related U.S. Application Data
(63) Continuation-in-part of application No. 10/586,863, filed on Jul. 21, 2006, now Pat. No. 8,829,048.

(30) Foreign Application Priority Data
Jan. 21, 2004 (IT) MI2004A 000069

(51) Int. Cl. A61K 31/232; A61K 9/00; A61K 9/48

(52) U.S. Cl. CPC A61K 31/232; A61K 9/4825; A61K 9/0053
USPC 514/549

(57) ABSTRACT

The use is described of a composition comprising either a) alpha-linolenic acid (ALA, C18:3 n-3) or b) docosahexaenoic acid (DHA, C22:6 n-3) or c) DHA in admixture with eicosapentaenoic acid (EPA, C20:5 n-3), in a ratio of 1:0.5 to 1:1.7, respectively, and/or the pharmaceutically acceptable derivatives and/or precursors thereof; either a) or b) or c) being in a concentration not lower than 70% by weight of the total fatty acids weight in the composition, for the preparation of a drug for the prevention and/or treatment of the disturbances of the central nervous system (CNS) such as epilepsy, schizophrenia, bipolar (manic-depressive illness) and unipolar (major depression) psychiatric disorders, and by degenerative Alzheimer's disease and related forms of dementia.

### CROSS-REFERENCE TO RELATED APPLICATIONS

This application is a continuation-in-part application of U.S. Nonprovisional application Ser. No. 10/586,863, filed Jul. 21, 2006, which is a 371 National Stage application of PCT Application No. PCT/EPOS/00522, filed Jan. 20, 2005, which claims the benefit of and priority to Italian Application No. MI2004A 000069, filed Jan. 21, 2004, all of which are hereby incorporated by reference in their entireties, for all purposes, herein.

The invention refers to the use of highly concentrated compositions of selected n-3 fatty acids for the treatment of central nervous system disturbances. In particular, the invention concerns the use of a composition comprising either alpha-linolenic acid (ALA, C18:3 n-3) or docosahexaenoic acid (DHA, C22:6 n-3) or DHA in admixture with eicosapentaenoic acid (EPA, C20:5 n-3) and/or the pharmaceutically acceptable derivatives and/or precursors thereof, in a high concentration, for the preparation of a drug for the prevention and/or treatment of the disturbances of the central nervous system (CNS), both of psychiatric relevance and of neurological type.

The expression “central nervous system disturbances” is commonly meant to indicate the cluster of convulsive symptoms usually included in the so-called epileptic syndromes, as well as to the most severe psychiatric disorders, represented by the various schizophrenia forms, by the manic-depressive syndromes, by the severe depression, and by the Alzheimer's disease and the related forms of dementia.

The term epilepsy refers to disorders of brain function characterized by periodic and unpredictable occurrence of seizures. Such seizures are constituted by transient alterations of behaviour caused by disordered, synchronous and rhythmic firing of neuronal brain populations, not induced by evident provocation.

These seizures are thought to arise from disorders of cerebral cortex, not involving other CNS structures, and their behavioural manifestations is determined by the functions served by the involved cortical site. For instance, a seizure arising from the motor cortex will induce f.i. clonic jerking of the body muscles controlled by this same region of the cortex. Epileptic seizures are defined partial, when beginning focally in a cortical site, or generalized seizures, when involving both hemispheres. A partial seizure is defined simple, if associated with preservation of consciousness, or complex in the opposite case, often due to impairment of the temporal lobe. A typical generalized epileptic seizure includes absence and tonic-clonic convulsions.

Schizophrenia is caused by a chemical imbalance in the brain induced, in its turn, by triggering causes of genetic or environmental origin (autoimmune diseases, infections during development, psychological trauma, etc.), involving—among other effects—overproduction of dopamine. There are several categories of the disease: paranoid, catatonic, disorganised, undifferentiated schizophrenia.

Patients start to get a great variety of symptoms, which anyway can reveal themselves into two typical forms: negative symptoms, such as withdrawal, apathy, depression, blunted emotions, and positive symptoms, such as hallucinations, misunderstanding of reality and of perception, disordered thinking and speech. The appearance of the disease is early, but diagnosis is complex and can take very long times.

The older drugs, typical neuroleptic drugs, essentially agents blocking cerebral dopamine, are however poorly selective and associated with heavy side effects on dopamine-related functions, including severe extrapyramidal effects, like unusual and involuntary body movements (dyskinesias, tardive dyskinesias), restlessness (alkathesia), muscle spams (dystonia), as well as impairment of cognition, reduced libido, etc; further, these drugs while moderately effective in treating positive symptoms, are quite unsuccessful on negative symptoms, as depression and apathy.

The more recent drugs, so-called atypical drugs, have a broader action spectrum and less side effects, such as the arising of involuntary movements, but are unavailable in under-developed countries because of their expensiveness, and are not free anyway from other effects, even highly risly (prolongation of QTC interval of ECG, weight gain, diabetic symptoms).

The syndromes of bipolar disturbances (manic-depressive disorders) and of severe unipolar depression (major depression) constitute the more severe disorders of mood or affect. They usually include disordered autonomic functioning (i.e., altered activity rhythms, sleep and appetite) and behaviour, as well as persistent abnormalities of mood and increased risk of self-destruction or suicide.

Alzheimer's disease, as other degenerative disease of CNS, all induced by progressive loss of neurons from specific regions of the brain, is characterized by marked atrophy of the cerebral cortex and loss of cortical, sub-cortical and hippocampal neurons; a parallel reduction of neurotransmitters has also been evidenced, in particular of acetylcholine, which has given rise to the cholinergic hypothesis of the disease and led to the few drugs of some limited effectiveness.

The disease produces a progressive impairment of the cognitive abilities, which is typical—but not exclusive—of the elder subject. The disease appears first as an impairment of short-term memory, but as the condition progresses additional cognitive abilities are impaired, such as the ability to calculate, exercise visual-spatial skills, and use common objects (ideo-motor apraxia), and the illness reveals itself in various forms of dementia. Later on, death often comes up from a complication of immobility, such as pneumonia.

For all mentioned pathologies, there are not valid pharmacological and clinical treatments, able to modify the progression of the disease. In all cases only symptomatic treatments are adopted, only able to alleviate the symptomatology and, if endowed with some efficacy, only effective on a very limited number of patients: for instance the standard treatments in the depression permit to obtain until a maximum of 50% reduction of score in the evaluation scale in two thirds of patients, while improvements in schizophrenia are obtained in the order of 20-30%, and treatments in the Alzheimer's disease result to be only ineffective palliatives.

Some therapeutic effects of the n-3 polyunsaturated fatty acids are already well known. For instance, IT 1,235,879, U.S. Pat. No. 5,502,077 and U.S. Pat. No. 5,656,667 disclose their effect on multiple risk factors for cardiovascular illnesses, as hypertriglyceridemia, hypercholesterolemia and hypertension.

EP-A-0409903 describes the preparation of high concentration mixtures of EPA and DHA or their esters, useful in the treatment of hyperlipemia, thrombosis, myocardial infarct, platelet hyperaggregation and related vascular pathologies, as well as of acute and chronic inflammations, autoimmune syndromes, and in the tumour prevention. DHA, which is contained in high concentration in the retina, is also active on the functionality of sight, on ceroidosis and on learning and ageing processes.

WO 00/48592 discloses the use of mixtures of EPA and DHA ethyl esters for the secondary prevention of “sudden death” in patients who have already suffered a myocardial infarct. It also results in the scientific literature that the n-3 polyunsaturated acids, particularly DHA, are contained in high concentration in the cerebral cortex (much less in the white matter), according to O'Brien J S and Sampson E L, J. Lipid Res. 6, 545, 1965, in the retina (Anderson R E, Exp. Eye Res. 10, 339, 1970), in the testis and sperm (Poulos A et al., Comp. Biochem. Physiol. 46B, 541, 1975) of all mammals, including human beings.

DHA is therefore one of the most abundant components of the brain's structural lipids, in which its presence can derive only from direct ingestion or by synthesis from the dietary precursor, i.e. alpha-linolenic acid (ALA).

Among others, Neuringer M et al., J. Clin. Invest. 73, 272, 1984; Proc. Natl. Acad. Sci. USA 83, 4021, 1986, suggest that n-3 fatty acids are essential for a normal prenatal and postnatal development of retina and brain.

EP-A-0347056 discloses the use of gamma-linolenic acid (GLA, C18:3 n-6) and higher n-6 acids, and of stearidonic acid (SA, C18:4 n-3) and higher n-3 acids, for the preparation of a drug for treatment of tardive dyskinesias.

EP-A-0599576 describes the use of a combination of arachidonic acid (AA, C20:4 n-6) and DHA, acids belonging to the n-6 and n-3 series respectively, to obtain a drug effective on the negative syndrome of schizophrenia, in subjects with low levels of the two acids in the cell membranes.

U.S. Pat. No. 6,331,568 discloses a method for treating schizophrenia by administration of EPA or SA, two n-3 acids, and optionally of n-6 acids. The compositions therein disclosed show a ratio of EPA or SA to DHA of not less than 3:1, 4:1, or more.

WO 00/44 361 discloses a pharmaceutical preparation containing at least 90% or more of EPA, and less than 5% of DHA for uses similar to the ones of the documents just above discussed.

U.S. Pat. No. 5,120,763 and EP 0366480 disclose a composition containing 13.0-27.5% of linolenic acid (i.e. ALA, C18:3 n-3) and 87.0-72.5% of linoleic acid (i.e. LA, C18:2 n-6), useful in the treatment of Alzheimer's disease and related syndromes, while U.S. Pat. No. 5,468,776 describes the same components in the more limited range of 16.7-22.2% and 83.3-77.8% respectively.

Although it is known to a certain degree that compositions comprising peculiar combinations of n-3 and/or n-6 fatty acids may have shown some effectiveness on pathologies such as schizophrenia or Alzheimer's disease, a clear indication on their activity against such pathologies cannot yet be taken from the prior art since the discussion in the scientific community is quite controversial and still open.

For instance, while Mellor et al., Human Psychopharmacology, 11, 39-46, 1996 disclosed the effectiveness of some n-3 acids, such as DHA, carrying out their experiments using a composition comprising EPA 18% and DHA 12%, in U.S. Pat. No. 6,331,568 there is pointed out that such ability has to be denied on the basis of both clinical control and for biochemical reasons.

It has been now surprisingly found that some other peculiar compositions comprising n-3 fatty acids in very high concentrations are effective for the prevention and/or treatment of various and severe disorders of the central nervous system, both of neurological type and of psychiatric pertinence, as for instance epilepsy and as schizophrenia, the manic—depressive disturbances and the major depression, as well as the degenerative neuronal disorders typical of Alzheimer's disease.

According to a first aspect the invention refers to the use of a composition comprising either

a) alpha-linolenic acid (ALA, C18:3 n-3) and/or the pharmaceutically acceptable derivatives and/or precursors thereof; or

b) docosahexaenoic acid (DHA, C22:6 n-3) and/or the pharmaceutically acceptable derivatives and/or precursors thereof; or

c) DHA in admixture with eicosapentaenoic acid (EPA, C20:5 n-3), in a ratio of 1:0.5 to 1:1.7, preferably of 1:0.9 to 1:1.5, respectively, and/or the pharmaceutically acceptable derivatives and/or precursors thereof; either a) or b) or c) being in a concentration not lower than 70% by weight of the total fatty acids weight in the composition, for the preparation of a drug for the prevention and/or treatment of the disturbances of the central nervous system (CNS), both of psychiatric relevance and of neurological type (e.g., neurological and/or psychiatric disturbances).

Preferably, the concentration in either a) or b) or c) is of 75% to 95%, in particular of 80% to 90%, most preferably of 85% (as a mean value). The ratio of DHA to EPA in c) can be, for example, 1:0.9 to 1:1.5.

Among the disturbances of CNS which can be prevented and/or treated according to the invention, there are epilepsy (showing partial and/or generalized seizures or simple and/or complex seizures), schizophrenia (showing negative and/or positive symptoms and being either paranoid or catatonic or disorganised or undifferentiated schizophrenia), manic-depressive syndrome, major depression (including disorders of mood, behaviour and autonomic functions correlated to activity, sleep and appetite), and Alzheimer's disease (including the various related forms of dementia).

In a preferred embodiment, the drug suitable for the use of the invention comprises essentially DHA ethyl ester and EPA ethyl ester.

Yet, according to another preferred embodiment, the composition can also comprise at least another n-3 and/or n-6 polyunsaturated and/or monounsaturated and/or saturated fatty acid, in particular the composition can comprise at least two other n-3 and/or n-6 polyunsaturated and/or monounsaturated and/or saturated fatty acids, in any ratio among themselves; the other n-3 and/or n-6 polyunsaturated and/or monounsaturated and/or saturated fatty acids are in a concentration of lower or equal to 30%.

Preferred ALA, DHA, and EPA derivatives are selected among C₁-C₃ alkyl esters (preferably ethyl esters), glyceride mono-, di-, tri-esters, and salts with pharmaceutically acceptable bases, like for instance sodium hydroxide and potassium hydroxide, aminoalcohols as ethanolamine and choline, basic aminoacids as arginine and lysine. “Precursor” is herein meant to indicate any compound able to lead to ALA, DHA and EPA through in vivo transformations, f.i. through metabolic processes.

Also the compositions reported in the European Pharmacopoeia 2000, Eu. Ph. 2000, having a content of not less than 80% of the mixture of the ethyl esters of EPA and DHA (not less than 40% and 34%, respectively) and not less than 90% total ethyl esters of n-3 polyunsaturated fatty acids, will be suitable for the use of the present invention.

All the above mentioned compositions, as well as the pharmaceutical preparations which can be derived therefrom, can be prepared according to methods known to the expert in the field, as f. i. those described in U.S. Pat. No. 5,130,061, WO 89/11 521, IT 1 235 879, DE 3 739 700, JP 02/25 447 (and others), herein incorporated by reference as far as their preparation is concerned.

Commonly, the composition suitable for the use of the invention can be obtained by extraction, concentration and purification processes starting from natural sources, typically from fish oils or other marine source as algae (for DHA and EPA), or even from vegetable oils, f.i. seed oils (typically for ALA), as well as by means of semi-synthetic transformation processes, when required.

Together with their efficacy to the aim of the pharmaceutical and therapeutic use of the invention, the above compositions show a very high clinical tolerance, almost free from any side-effect, with exclusion of some uncommon effect on the intestinal peristalsis, and can be obtained with low production costs from natural sources, which strongly helps their diffusion in low economic potential countries, differently from the poor availability of some totally synthetic drugs.

The drug suitable for the use of the invention is preferably administered by oral route, particularly in the form of soft jelly capsules; yet, the other typical administration routes, usual in the pharmaceutical technology, are not excluded. The dose per unit includes usually 100-1000 mg of the above specified n-3 polyunsaturated fatty acids and/or derivatives and/or precursors, preferably 300-1000 mg or better 500 mg or more often 1000 mg. The mean total dose is 0.1-5 g per day, even in intermittent administration, according to the need and advice of the physician, preferably 0.3-3 g per day or particularly 1-2 g/day. An effective dose meanly corresponds to 2-60 mg/kg/day.

Obviously, the drug suitable for the use of the invention can be administered also under other forms appropriate for the oral administration such as, for instance, hard oil-proof capsules or tablets wherein the fatty acids are pre-adsorbed on solid matrices.

It is also possible to use oily emulsions, syrups, drops, granulates in dispersing excipients, etc., as well as other forms able to guarantee a systemic absorption by means of other administration routes, f. i. sterile emulsions or solutions suitable for parenteral injective use, as it will be apparent to the man skilled in the art.

The drug suitable for the use of the present invention can be used alone, as a mono-therapy, or as a drug coadjuvant or auxiliary to at least another active principle or drug effective for the prevention and/or treatment of the disturbances of CNS, or can even be used in direct combination, including said at least another active principle or drug endowed with an activity similar or complementary or synergic to that one of the above defined drug suitable for the use of the invention.

Typical examples of such at least another active principle or drug to which the drug suitable for the use of the invention can be combined or can be auxiliary or coadjuvant by co-administration, are, without any limitative meaning:
in the treatment of epilepsy, carbamazepine, phenyloin, phenobarbital, primidone, valproate, gabapentin, lamotrigine, clonazepam, ethosuximide, and related structures;
in the treatment of schizophrenia, drugs of the group of phenothiazines, thioxanthenes, dibenzoazepines, butyrophenones, indolones, phenyl- and diphenylpiperidines, etc., among them the typical neuroleptic agents as chlorpromazine, thioridazine, haloperidol, sulpiride, and pimozide, and others, and the antipsychotic “atypical” agents as clozapine, quetiapine, olanzapine, sertindole, risperidone, ziprasidone, amisulpiride and others;
in the treatment of major depression and of manic-depressive illness, the antidepressant drugs of the group of tricyclic norepinephrine reuptake inhibitors as amitriptyline and others, of the group of serotonin reuptake inhibitors as fluoxetine, paroxetine, sertraline and others, of the group of monoamine oxidase (MAO) inhibitors as phenelzine and others, of the group of “atypical” drugs as bupropion and trazodone; the antimanic drugs as lithium salts; the drugs acting on mood and affect disorders as many antianxiety agents, including benzodiazepines and the above mentioned antidepressant and antimanic drugs as well as some anticonvulsants/antiepileptic drugs as carbamazepine and valproate;
in the treatment of Alzheimer's disease, among the few “approach” drugs, the precursors of acetylcholine, as choline and phosphatidylcholine, and the inhibitors of catabolic enzyme (acetylcholinesterase, AChE), as physostigmine, tacrine, donepezilrivastigmine and galantamine, as well as memantine, more recently adopted and endowed with a different mechanism of action.

The composition suitable for the use of the invention can also comprise a pharmaceutically acceptable diluent, and/or a vehicle, and/or a binder, and/or thickener, and/or a surfactant, as well as a lubricant, aromatizer, colorant, sweetener, stabilizer and the like, as it will be apparent to the man skilled in the art. Among the stabilizer agents, antioxidants, particularly tocopherol (vitamin E) and the like, as well as ascorbyl palmitate, hydroxytoluene, butylhydroxyanisole and the like known in the art, are particularly preferred.

As already illustrated above, the drug comprising either a) or b) or c) as above defined can be administered according to the invention either as a single drug or in fixed pharmaceutical combination with other known drugs already known to be used in the same pathologies, or even as substances coadjuvant to said known drugs, under separated administration.

According to another aspect, the invention relates to a method for prevention and/or treatment of CNS disturbances, as above illustrated, in a mammal in need thereof comprising administering to the mammal a therapeutically effective dose, preferably ranging from about 2 to 60 mg/kg of the mammal body weight per day, of a drug as above described.

In another aspect, a method for the treatment or prevention of at least one of manic-depressive syndrome and major depression includes administering to a subject (e.g., a subject in need thereof) a composition consisting essentially of, in a concentration expressed as % by weight of the total fatty acid weight in the composition, DHA ethyl ester >80 and EPA ethyl ester <15, wherein EPA+DHA ethyl esters >85. The subject may be an individual who is suffering from one or both of major depression and manic-depressive syndrome (e.g., an individual diagnosed with one or both of major depression and manic-depressive syndrome) and the composition is effective for delaying, alleviating, or reducing at least one symptom (e.g., one symptom, two symptoms, three symptoms, etc.) thereof in the subject. The subject may also be an individual who is susceptible to the disease (e.g., at risk of suffering from the disease, suspects they are suffering from the disease, an undiagnosed individual), and the composition is effective for delaying, alleviating, or reducing at least one symptom (e.g., one symptom, two symptoms, three symptoms, etc.) thereof in the subject. The composition can be administered by any suitable route, e.g., oral route, and the composition can be prepared in any suitable formulation, e.g., in the form of soft gelatine capsules. The composition can be administered, for example, at the dose of 0.1-5 g/day (e.g., a dose of 0.3-3 g/day, a dose of 1-2 g/day, etc.). In the method, the composition can be administered separately, as a coadjuvant or an auxiliary drug, from at least another drug effective for the treatment of at least one of manic-depressive syndrome and major depression. In the composition, the EPA ethyl ester can be 0% by weight or present in a trace amount.

In yet another aspect, a method for the treatment or prevention of manic-depressive syndrome includes administering to a subject (e.g., a subject in need thereof) a composition consisting essentially of, in a concentration expressed as % by weight of the total fatty acid weight in the composition, DHA ethyl ester >34 and EPA ethyl ester >40, wherein EPA+DHA ethyl esters >80, the total ethyl esters of n-3 acids being >90. The subject may be an individual who is suffering from manic-depressive syndrome (e.g., an individual diagnosed with manic-depressive syndrome) and the composition is effective for delaying, alleviating, or reducing at least one symptom (e.g., one symptom, two symptoms, three symptoms, etc.) thereof in the subject. The subject may also be an individual who is susceptible to the disease (e.g., at risk of suffering from the disease, suspects they are suffering from the disease, an undiagnosed individual), and the composition is effective for delaying, alleviating, or reducing at least one symptom (e.g., one symptom, two symptoms, three symptoms, etc.) thereof in the subject. The composition can be administered by any suitable route, e.g., oral route, and the composition can be prepared in any suitable formulation, e.g., in the form of soft gelatine capsules. The composition can be administered, for example, at the dose of 0.1-5 g/day (e.g., a dose of 0.3-3 g/day, a dose of 1-2 g/day, etc.). In the method, the composition can be administered separately, as a coadjuvant or an auxiliary drug, from at least another drug effective for the treatment of manic-depressive syndrome.

In a further aspect, a method for the treatment or prevention of one or more diseases or disorders such as (at least one of) manic depressive syndrome, major depression and Alzheimer's disease includes administering to a subject a composition consisting essentially of DHA ethyl ester and EPA ethyl ester. The subject may be an individual who is suffering from one of these diseases (e.g., an individual diagnosed with one or more of these diseases) and the composition is effective for delaying, alleviating, or reducing at least one symptom (e.g., one symptom, two symptoms, three symptoms, etc.) thereof in the subject. In one embodiment, the subject is susceptible to one or more of these diseases (e.g., at risk of suffering from the disease, suspects they are suffering from the disease, an undiagnosed individual), and administration of the composition delays, alleviates, or reduces at least one symptom (e.g., one or more symptoms). The composition can be administered by any suitable route, for example, by an oral route. In such a method, the composition may be in the form of soft gelatine capsules. In the method, the composition can be administered at a dose of 0.1-5 g/day (e.g., a dose of 0.3-3 g/day, a dose of 1-2 g/day, etc.). In one embodiment, the composition can be administered separately, as a coadjuvant or an auxiliary drug, from at least another drug effective for the treatment of one or more of these diseases.

In one aspect, a method for the treatment or prevention of Alzheimer's disease includes administering to a subject a composition that includes either

a) alpha-linolenic acid (ALA, C18:3 n-3) and/or the pharmaceutically acceptable derivatives and/or precursors thereof; or

b) docosahexaenoic acid (DHA, C22:6 n-3) and/or the pharmaceutically acceptable derivatives and/or precursors thereof; or

c) DHA in admixture with eicosapentaenoic acid (EPA, C20:5 n-3), in a ratio of 1:0.5 to 1:1.7, preferably of 1:0.9 to 1:1.5, respectively, and/or the pharmaceutically acceptable derivatives and/or precursors thereof; either a) or b) or c) being in a concentration not lower than 70% by weight of the total fatty acids weight in the composition, for the preparation of a drug for the prevention and/or treatment of the Alzheimer's disease. In this method, the concentration in either a) or b) or c) can be, for example, of 75% to 95%, in particular of 80% to 90%, or of 85% (as a mean value). The subject may be an individual who is suffering from Alzheimer's disease (e.g., an individual diagnosed with Alzheimer's disease) and the composition is effective for delaying, alleviating, or reducing at least one symptom (e.g., one symptom, two symptoms, three symptoms, etc.) thereof in the subject. In one embodiment, the subject is susceptible to Alzheimer's disease (e.g., at risk of suffering from the disease, suspects they are suffering from the disease, an undiagnosed individual), and administration of the composition delays, alleviates, or reduces at least one symptom (e.g., one or more symptoms). The composition can be administered by any suitable route, for example, by an oral route. In such a method, the composition may be in the form of soft gelatine capsules. In the method, the composition can be administered at a dose of 0.1-5 g/day (e.g., a dose of 0.3-3 g/day, a dose of 1-2 g/day, etc.). In one embodiment, the composition can be administered separately, as a coadjuvant or an auxiliary drug, from at least another drug effective for the treatment of Alzheimer's disease.

In another aspect of treating and/or preventing Alzheimer's disease, the method includes administering to a subject (e.g., a subject in need thereof) a composition consisting essentially of, in a concentration expressed as % by weight of the total fatty acid weight in the composition, one selected from:
a) DHA ethyl ester >30 and EPA ethyl ester >44, wherein EPA+DHA ethyl esters >80, the ethyl esters of other (C20, C21, C22) n-3 acids being >3; and
b) DHA ethyl ester >34 and EPA ethyl ester >40, wherein EPA+DHA ethyl esters >80, the total ethyl esters of n-3 acids being >90.

In one embodiment of this method of treating Alzheimer's disease, the composition consists essentially of, in a concentration expressed as % by weight of the total fatty acid weight in the composition, DHA ethyl ester >30 and EPA ethyl ester >44, wherein EPA+DHA ethyl esters >80, the ethyl esters of other (C20, C21, C22) n-3 acids being >3. In another embodiment of this method, the composition consists essentially of, in a concentration expressed as % by weight of the total fatty acid weight in the composition, DHA ethyl ester >34 and EPA ethyl ester >40, wherein EPA+DHA ethyl esters >80, the total ethyl esters of n-3 acids being >90. In yet another embodiment, the composition consists essentially of, in a concentration expressed as % by weight of the total fatty acid weight in the composition, DHA ethyl ester >80 and EPA ethyl ester <15, wherein EPA+DHA ethyl esters >85. In all of these embodiments, the subject may be an individual who is suffering from Alzheimer's disease (e.g., an individual diagnosed with Alzheimer's disease) and the composition is effective for delaying, alleviating, or reducing at least one symptom (e.g., one symptom, two symptoms, three symptoms, etc.) thereof in the subject. The subject may be an individual who is susceptible to Alzheimer's disease (e.g., at risk of suffering from the disease, suspects they are suffering from the disease, an undiagnosed individual), and administration of the composition delays, alleviates, or reduces at least one symptom (e.g., one or more symptoms). The composition can be administered by any suitable route, for example, by an oral route. In such a method, the composition may be in the form of soft gelatine capsules. In the method, the composition can be administered at a dose of 0.1-5 g/day (e.g., a dose of 0.3-3 g/day, a dose of 1-2 g/day, etc.). In one embodiment, the composition can be administered separately, as a coadjuvant or an auxiliary drug, from at least another drug effective for the treatment of Alzheimer's disease.

In still another aspect, a food product for treating or preventing at least one of manic depressive syndrome and major depression in a subject includes a composition consisting essentially of, in a concentration expressed as % by weight of the total fatty acid weight in the composition, DHA ethyl ester >80 and EPA ethyl ester <15, wherein EPA+DHA ethyl esters >85. The composition is effective for delaying, alleviating, or reducing at least one symptom (e.g., one symptom, two symptoms, three symptoms, etc.) thereof. The subject may be an individual who is susceptible to (e.g., at risk of suffering from the disease, suspects they are suffering from the disease, an undiagnosed individual) at least one of manic depressive syndrome and major depression, and the composition is effective for delaying, alleviating, or reducing at least one symptom thereof when ingested or consumed by the subject. The subject may be an individual who is suffering from one or both of major depression and manic-depressive syndrome (e.g., an individual diagnosed with one or both of major depression and manic-depressive syndrome) and the composition is effective for delaying, alleviating, or reducing at least one symptom (e.g., one symptom, two symptoms, three symptoms, etc.) thereof in the subject. In a typical embodiment, ingestion or consumption of the food product delays, alleviates or reduces symptoms associated with major depression and/or manic-depressive syndrome in the subject (e.g., a subject susceptible to or at risk of one or both of these diseases, a subject diagnosed and/or suffering from one or both of these diseases). Examples of such food products include beverages, cereals, dairy products, fats and oils, dietetic products, and food supplements. The composition can be present in any suitable amount, typically in the range of about 0.05 gram to about 5 gram per serving of the food product. In one embodiment, the composition is present at the dose of 0.1-5 gram (e.g., a dose of 0.1-5 g/day, a dose of 0.3-3 g/day, a dose of 1-2 g/day, etc.). In another embodiment, the composition is present at the dose of about 0.1 gram to about 2.0 gram.

In yet another aspect, a food product for treating or preventing manic depressive syndrome in a subject includes a composition consisting essentially of, in a concentration expressed as % by weight of the total fatty acid weight in the composition, DHA ethyl ester >34 and EPA ethyl ester >40, wherein EPA+DHA ethyl esters >80, the total ethyl esters of n-3 acids being >90. The composition is effective for delaying, alleviating, or reducing at least one symptom thereof. The subject can be an individual who is susceptible to (e.g., at risk of suffering from) manic depressive syndrome, and the composition is effective for delaying, alleviating, or reducing at least one symptom thereof when ingested or consumed by the subject. The subject may be an individual who is suffering from manic-depressive syndrome (e.g., an individual diagnosed with manic-depressive syndrome) and the composition is effective for delaying, alleviating, or reducing at least one symptom (e.g., one symptom, two symptoms, three symptoms, etc.) thereof in the subject. In a typical embodiment, ingestion or consumption of the food product delays, alleviates or reduces symptoms associated with manic-depressive syndrome in a subject (e.g., a subject susceptible to or at risk of manic-depressive syndrome, a subject diagnosed with and/or suffering from manic-depressive syndrome). Examples of such food products include beverages, cereals, dairy products, fats and oils, dietetic products, and food supplements. The composition can be present in any suitable amount, typically in the range of about 0.05 gram to about 5 gram per serving (e.g., per dose) of the food product (e.g., 0.1-5 g/day, 0.3-3 g/day, 1-2 g/day, etc., in one or more doses or servings).

According to another aspect, the invention includes a food product that includes, in an amount effective for treating or preventing Alzheimer's disease in a subject (e.g., a subject susceptible to or at risk of Alzheimer's disease, a subject diagnosed with and/or suffering from Alzheimer's disease), any of the compositions described above, e.g., a composition consisting essentially of, in a concentration expressed as % by weight of the total fatty acid weight in the composition, DHA ethyl ester >34 and EPA ethyl ester >40, wherein EPA+DHA ethyl esters >80, the total ethyl esters of n-3 acids being >90; a composition consisting essentially of, in a concentration expressed as % by weight of the total fatty acid weight in the composition, DHA ethyl ester >80 and EPA ethyl ester <15, wherein EPA+DHA ethyl esters >85; a composition consisting essentially of DHA ethyl ester and EPA ethyl ester, etc. For example, the food product can include, in a quantity of at least 0.05 g per serving of the food product (e.g., 0.05 g to about 5 g, 0.1-5 g/day, 0.3-3 g/day, 1-2 g/day, etc. in one or more servings), a composition consisting essentially of, in a concentration expressed as % by weight of the total fatty acid weight in the composition, DHA ethyl ester >34 and EPA ethyl ester >40, wherein EPA+DHA ethyl esters >80, the total ethyl esters of n-3 acids being >90. As described above, the composition is effective for delaying, alleviating or reducing one or more symptoms associated with Alzheimer's disease.

In another embodiment, a food product for the treatment or prevention of one or more of (at least one of) manic depressive syndrome, major depression and Alzheimer's disease includes a composition consisting essentially of DHA ethyl ester and EPA ethyl ester.

In yet a further aspect, a method for treating or preventing a disease including major depression and manic-depressive syndrome in a subject includes administering to the subject a therapeutically effective dose of a product for oral ingestion that includes a composition consisting essentially of, in a concentration expressed as % by weight of the total fatty acid weight in the composition, DHA ethyl ester >80 and EPA ethyl ester <15, wherein EPA+DHA ethyl esters >85. The product for oral ingestion may be, for example, a food product. Typically, the composition is effective for at least one of: delaying, alleviating, and reducing at least one symptom (e.g., one symptom, two symptoms, three symptoms, etc.) associated with the disease. The subject may be an individual who is suffering from one or both of major depression and manic-depressive syndrome (e.g., an individual diagnosed with one or both of major depression and manic-depressive syndrome) and the composition is effective for delaying, alleviating, or reducing at least one symptom (e.g., one symptom, two symptoms, three symptoms, etc.) thereof in the subject. The subject may also be an individual who is susceptible to the disease (e.g., at risk of suffering from the disease, suspects they are suffering from the disease, an undiagnosed individual), and the composition is effective for delaying, alleviating, or reducing at least one symptom (e.g., one symptom, two symptoms, three symptoms, etc.) thereof in the subject. In a typical embodiment, the composition is present in the range of about 0.05 gram to about 5 gram per serving (e.g., about 0.1 gram to about 2.0 gram per serving) of the food product.

In another aspect, a method for treating or preventing manic-depressive syndrome in a subject includes administering to the subject a therapeutically effective dose of a product for oral ingestion that includes a composition consisting essentially of, in a concentration expressed as % by weight of the total fatty acid weight in the composition, DHA ethyl ester >34 and EPA ethyl ester >40, wherein EPA+DHA ethyl esters >80, the total ethyl esters of n-3 acids being >90. The product for oral ingestion may be, for example, a food product. The composition is effective for delaying, alleviating, or reducing at least one symptom associated with manic-depressive syndrome. The subject may be an individual who is suffering from manic-depressive syndrome (e.g., an individual diagnosed with manic-depressive syndrome) and the composition is effective for delaying, alleviating, or reducing at least one symptom (e.g., one symptom, two symptoms, three symptoms, etc.) thereof in the subject. The subject may also be an individual who is susceptible to the disease (e.g., at risk of suffering from the disease, suspects they are suffering from the disease, an undiagnosed individual), and the composition is effective for delaying, alleviating, or reducing at least one symptom (e.g., one symptom, two symptoms, three symptoms, etc.) thereof in the subject. In a typical embodiment, the composition is present in the range of about 0.05 gram to about 5 gram per serving (e.g., about 0.1 gram to about 2.0 gram per serving) of the food product.

In yet another aspect, a method for treating and/or preventing Alzheimer's disease includes administering to the subject a therapeutically effective dose of a product for oral ingestion that includes, in an amount effective for treating or preventing Alzheimer's disease in a subject (e.g., a subject susceptible to or at risk of Alzheimer's disease, a subject diagnosed with and/or suffering from Alzheimer's disease), any of the compositions described above, e.g., a composition consisting essentially of, in a concentration expressed as % by weight of the total fatty acid weight in the composition, DHA ethyl ester >34 and EPA ethyl ester >40, wherein EPA+DHA ethyl esters >80, the total ethyl esters of n-3 acids being >90; a composition consisting essentially of, in a concentration expressed as % by weight of the total fatty acid weight in the composition, DHA ethyl ester >80 and EPA ethyl ester <15, wherein EPA+DHA ethyl esters >85; a composition consisting essentially of DHA ethyl ester and EPA ethyl ester, etc. For example, the product for oral ingestion can include, in a quantity of at least 0.05 g per serving of the product for oral ingestion (e.g., 0.05 g to about 5 g, 0.1-5 g/day, 0.3-3 g/day, 1-2 g/day, etc.), a composition consisting essentially of, in a concentration expressed as % by weight of the total fatty acid weight in the composition, DHA ethyl ester >34 and EPA ethyl ester >40, wherein EPA+DHA ethyl esters >80, the total ethyl esters of n-3 acids being >90. As described above, the composition is effective for delaying, alleviating or reducing one or more symptoms associated with Alzheimer's disease. In some embodiments, the product for oral ingestion is a food product.

According to another aspect, the present invention relates to a method for at least one of: delaying, alleviating and reducing symptoms associated with major depression, manic-depressive illness and/or Alzheimer's disease, as described above, in a subject susceptible to or at risk of one or more such diseases including administering to the subject a therapeutically effective dose of a food product as described above.

In yet another aspect, a product for oral ingestion for the treatment or prevention of at least one of manic-depressive syndrome and major depression in a subject includes a composition consisting essentially of, in a concentration expressed as % by weight of the total fatty acid weight in the composition, DHA ethyl ester >80 and EPA ethyl ester <15, wherein EPA+DHA ethyl esters >85. The subject may be an individual who is suffering from one or both of major depression and manic-depressive syndrome (e.g., an individual diagnosed with one or both of major depression and manic-depressive syndrome) and the composition is effective for delaying, alleviating, or reducing at least one symptom (e.g., one symptom, two symptoms, three symptoms, etc.) thereof in the subject. The subject may also be an individual who is susceptible to the disease (e.g., at risk of suffering from the disease, suspects they are suffering from the disease, an undiagnosed individual), and the composition is effective for delaying, alleviating, or reducing at least one symptom (e.g., one symptom, two symptoms, three symptoms, etc.) thereof in the subject.

In a further aspect, a product for oral ingestion for the treatment or prevention of manic-depressive syndrome in a subject including a composition consisting essentially of, in a concentration expressed as % by weight of the total fatty acid weight in the composition, DHA ethyl ester >34 and EPA ethyl ester >40, wherein EPA+DHA ethyl esters >80, the total ethyl esters of n-3 acids being >90. The subject may be an individual who is suffering from manic-depressive syndrome (e.g., an individual diagnosed with manic-depressive syndrome) and the composition is effective for delaying, alleviating, or reducing at least one symptom (e.g., one symptom, two symptoms, three symptoms, etc.) thereof in the subject. The subject may also be an individual who is susceptible to the disease (e.g., at risk of suffering from the disease, suspects they are suffering from the disease, an undiagnosed individual), and the composition is effective for delaying, alleviating, or reducing at least one symptom (e.g., one symptom, two symptoms, three symptoms, etc.) thereof in the subject.

According to another aspect, the invention includes a product for oral ingestion for the treatment or prevention of Alzheimer's disease that includes, in an amount effective for treating or preventing Alzheimer's disease in a subject, any of the compositions described above, e.g., a composition consisting essentially of, in a concentration expressed as % by weight of the total fatty acid weight in the composition, DHA ethyl ester >34 and EPA ethyl ester >40, wherein EPA+DHA ethyl esters >80, the total ethyl esters of n-3 acids being >90; a composition consisting essentially of, in a concentration expressed as % by weight of the total fatty acid weight in the composition, DHA ethyl ester >80 and EPA ethyl ester <15, wherein EPA+DHA ethyl esters >85; a composition consisting essentially of DHA ethyl ester and EPA ethyl ester, etc. In this embodiment, the product for oral ingestion can include a quantity of at least 0.05 g of the composition per serving of the product for oral ingestion (e.g., 0.05 g to about 5 g, 0.1-5 g/day, 0.3-3 g/day, 1-2 g/day, etc.). As described above, the composition is effective for delaying, alleviating or reducing one or more symptoms associated with Alzheimer's disease. The subject may be an individual who is suffering from Alzheimer's disease (e.g., an individual diagnosed with Alzheimer's disease) and the composition is effective for delaying, alleviating, or reducing at least one symptom (e.g., one symptom, two symptoms, three symptoms, etc.) thereof in the subject. The subject may also be an individual who is susceptible to the disease (e.g., at risk of suffering from the disease, suspects they are suffering from the disease, an undiagnosed individual), and the composition is effective for delaying, alleviating, or reducing at least one symptom (e.g., one symptom, two symptoms, three symptoms, etc.) of Alzheimer's disease in the subject.

What is meant by the terms “preventing” and “prevention” is well known in the art, and can include, for example, delaying symptoms (e.g., delaying or prolonging the first occurrence of one or more symptoms of a disease in a subject), alleviating symptoms (e.g., decreasing the severity of a re-occurring symptom(s) of a disease), and/or reducing symptoms (e.g., reducing the number of re-occurrences of a symptom(s) of a disease).

The phrase “administering to the subject a therapeutically effective dose of a food product” encompasses a subject ingesting (consuming) the food product, e.g., a subject self-administering the food product.

As described herein, the product for oral ingestion can be, for example, a food product. A food product as described herein can be any food, preferably one chosen from beverages (e.g. fruit-based beverages, energy drinks, soft drinks, soy milk), cereals (e.g. breads, breakfast cereals, pasta, rice, noodles, biscuits/cookies, savory snacks, bread-based snacks, nutritional bars), dairy products (e.g. milk, yogurt or yogurt-type/based foods, cheese, ice-cream), fats and oils (e.g. margarines, salad dressings, mayonnaise, edible oils), dietetic products (e.g. milk powder, infant formula, slimming powders, maternal nutrition, medical nutrition, sports nutrition), food supplements (e.g. in the form of a granular product, microencapsulates, nanoparticles), etc.

Such food products can be prepared by the incorporation of the above-mentioned compositions, in liquid or dry form, as well as in the form of nanoparticles, microencapsulates and the like, in a suitable concentration, in the industrial production of the food product. For example, in the case of a yogurt, the composition can be mixed in the yogurt after its preparation, prior to packaging; in the case of a nutritional bar, it can be incorporated in the bar mix before it is formed and packaged.

With reference to the microencapsulates, these are manufactured according to well known techniques in the field, e.g. by applying several numbers of coatings to a certain amount of the composition in the liquid form. The advantage of using microencapsulates over the oily composition in the industry, is that of reducing the smell and taste of the oily composition in the food. The microencapsulates are suitable to be mixed or incorporated into a food during the industrial production thereof or at any time before intake of such food.

In the case of nanoparticles, they are manufactured according to well known techniques in the field, e.g. by the nano-sized self-assembled structural liquid (NSSL) technology. The advantage of using nanoparticles over the oily composition in the industry, is that of reducing the smell and taste of the oily composition in the food. This product is suitable to be mixed or incorporated into a food during the industrial production thereof or at any time before intake of such food.

In the case of the granular product, it is manufactured according to well known techniques in the field, e.g. granulation starting from a powder product, in the presence of one or more additional ingredients such as binders, diluents, surfactants, fillers, etc. This product is suitable to be dissolved or mixed into a food at any time before intake of such food.

The above-listed food products can be refrigerated, frozen or stable at room temperature, to be stored and/or served or used dry, frozen, cold or in the form of a hot or cold drink or food.

Regarding the expressions “a subject susceptible to” and “a subject at risk of” one or more of the above diseases, what is typically meant is:
a) In the case of manic-depressive syndrome, the occurrence of the following: a period of at least one week of persistently elevated, expansive or irritable mood; the presence during such period of one or more of: inflated self-esteem or grandiosity; decreased need for sleep; being more talkative than usual; flight of ideas or subjective experience that thoughts are racing.
b) In the case of major depression, a period of at least two weeks with the occurrence of one or more of the following: depressed mood for most of the day, nearly every day; markedly diminished interest or pleasure in all, or almost all, activities—most of the day, nearly every day; insomnia or hypertension nearly every day; psychomotor agitation or retardation nearly every day; fatigue or loss of energy nearly every day; feelings of worthlessness or excessive or inappropriate guilt nearly every day.
c) In the case of Alzheimer's disease, the occurrence of one or more of the following: frequent memory loss that disrupts daily life, repeated questions, mild coordination problems, depression and apathy, difficulty completing familiar tasks at home, at work or at leisure, confusion with time or place, new problems with words in speaking or writing, misplacing things and losing the ability to retrace steps.
d) A person who has a family history of one or more of the diseases.

The administration of the food product of the invention can be carried out, for example, by replacement of a normally consumed food product (for example a yogurt or a fruit juice at breakfast) with a corresponding food product of the present invention. The administration of such food products to a subject susceptible to or at risk of one of the diseases described herein, as well as to a subject diagnosed with and/or suffering from one of these diseases, can have the effect of reducing the number of re-occurrences of any symptoms of the disease, of delaying the onset of the disease and/or of reducing the severity of the disease. Generally, a subject as described herein is a human being (e.g., human infant, human adolescent, human adult).

In a typical embodiment, the quantity of composition present per serving of a food product for consumption by an adult subject is between about 0.1 g and about 2.0 g (e.g., about 1.0 g. In another typical embodiment, the quantity of composition present per serving of a food product for consumption by an infant subject is between about 0.05 g and 1 g. A serving is generally defined as the portion of food used as a reference in the nutrition label of that food.

All publications, patent applications, and patents mentioned herein are incorporated by reference in their entirety. In the case of conflict, the present specification, including definitions, will control. The particular embodiments discussed below are illustrative only and not intended to be limiting. The following examples illustrate the invention without limiting it.

### EXAMPLE 1

A few compositions of the invention are illustrated in the following Table and can be prepared according to the methods described in U.S. Pat. No. 5,130,061 (compositions A, C, D, E), IT 1235879 (composition B), WO 89/11521 (compositions F, H, I) and DE 3739700 (composition G), and are anyway easily available even using other preparative methods (JP 02/25447 and several others).

The quantities indicated in the Table express percentages by weight on the total weight of fatty acids. Other n-3 acids, as well as n-6 unsaturated acids, having different length and/or unsaturation degree, monounsaturated and saturated, can be present in limited quantities. Antioxidant: alpha-tocopherol (mean <0.3%; even much higher concentrations can be used).

| | A 1) | B 1) | C 1) | D 1) | E 1) | F 1) | G 1) | H 2) | I 3) |
|---|---|---|---|---|---|---|---|---|---|
| EPA | >40 | >44 | >40 | >35 | >30 | <15 | | >40 | >50 |
| DHA | >34 | >30 | >34 | >30 | >35 | >80 | | >30 | >30 |
| EPA + DHA | >85 | >80 | >80 | >70 | >70 | >85 | | >80 | >80 |
| Esters 4) | | >3 | | | | | | | |
| Total n-3 esters 5) | | | >90 | | | | | | |
| ALA | | | | | | | >70 | | |

1) ethyl esters;
2) free acids;
3) sodium salts;
4) ethyl esters of other (C20, C21, C22) n-3 acids;
5) total ethyl esters of n-3 acids.

### EXAMPLE 2

The compositions of the following Table, relative to soft gelatin capsules containing 1 g ethyl esters of polyunsaturated fatty acid, were prepared by methods known in the art.

| | A (mg) | B (mg) | C (mg) |
|---|---|---|---|
| EPA 1) | 525 | | >400 |
| DHA 1) | 315 | | >340 |
| EPA + DHA 1) | | 850 | >800 |
| Total n-3 1) | | | >900 |
| d-tocopherol | 4 Units | | 4 Units |
| d,1-tocopherol | | 0.3 | |
| Gelatine | 246 | | 246 |
| Gelatine succinate | | 233 | |
| Glycerol | 118 | 67 | 118 |
| RIO | 2.27 | | 2.27 |
| YIO | 1.27 | | 1.27 |

| | |
|---|---|
| SHB + SPHB | 1.09 + 0.54 |

1) ethyl esters;
RIO: red iron oxide;
YIO; yellow iron oxide;
SHB: sodium p.hydroxybenzoate;
SPHB: sodium propyl p.hydroxybenzoate.

### EXAMPLES 3-6

### Pharmacological Activity

The effectiveness of the composition suitable for the use of the invention in the prevention and/or treatment of CNS disturbances as those above described, as well as of the possibility of their pharmaceutical and clinical use, has been demonstrated following several pharmacological tests which permitted a wide testing on small size animals (mice, rats), without the ethical implications proper of testing in humans.

A first model of experiment evaluated the protection against epileptic seizures induced by direct application of iron chloride to cerebral cortex; a second model examined the protective effect against a known convulsant chemical agent (pentylenetetrazol); a third model verified the effect on the induction of epileptic seizures provoked by repeated sub-convulsive dose administration of the same chemical agent and a fourth experimental model evaluated the effect protective on the anomalous behaviour induced, in the form of irregular jumping, by the administration of dizocilpine, an analogue of phencyclidine which binds similarly to N-methyl-D-aspartate (NMDA) receptors provoking its hypo-function and inducing schizophreniform psychosis.

In some of such tests, attention has been particularly addressed to detect any activity strengthening or coadjuvant to that of other known drugs.

Male Sprague-Dawley albino rats, about two months of age and 200-240-g weight, were used in the experiments of Examples 3-5. The animals were housed at an average temperature of about 22° C. and an average relative humidity of 40-50%, with artificial daily light cycles of 12 hours. In example 6, male Swiss mice weighing 22-30 g, housed in similar room conditions were used.

### EXAMPLE 3

Two groups of 15 rats were treated for 2 weeks by intraperitoneal (i.p.) route with 50 mg/kg of composition B (Example 1) containing >80% EPA and DHA ethyl esters (group 1) and with saline solution (group 2, control), respectively. At the end, all rats received a dose of 5 microlitres of 100 mM solution of FeCl₃, directly injected through unilateral left-side cannula into the anterior amygdala area. The administration of FeCl₃ into the cerebral cortex or amygdala-hippocampus complex is able to induce an epileptic focus, according to Willmore L. J., Science, 200, 1501, 1978. By prolonged direct monitoring, the number of animals protected or subject to major motor epileptic seizures are determined, as evidenced by tonic-clonic contractions of the limbs, trunk and head, lack of straightening reflex, saliva and blood discharge from the mouth.

Results: animals responding with seizures:
3/15 (group 1, treated)
14/15 (group 2, control)

### EXAMPLE 4

In a preliminary experiment, the dose effective on 50% of study strain rats of a known epileptogenic agent (pentylenetetrazol) injected by intraperitoneal route, has been determined, obtaining an ED50 value of approximately 70-75 mg/kg i.p.

Four groups of 10 rats each were treated daily for 2 weeks, by i.p. route, with 50 mg/kg of a composition of n-3 fatty acids having >85% of EPA and DHA ethyl esters according to Example 1, composition A (group 1), with 5 mg/kg of a known antiepileptic drug represented by clonazepam (group 2), with the same doses in combination (group 3) and with saline solution (group 4, control). At the end, all the groups were treated with 100 mg/kg i.p. of pentylenetetrazol, and the animals underwent the following control exams: 1) length of latency period to the first major motor seizures (tonic-clonic contractions of the limbs, trunk and head; falling; saliva and blood discharge from the mouth); 2) number of rats responding with major motor seizures (or protected from seizure); 3) mean duration of the major motor seizure (MMS); 4) number of rats presenting minor clonic contractions (MCC), such as a sudden flexion of the forelegs or extension of the rear legs; 5) number of rats who died within 20 minutes or 5 hours after pentylenetetrazol injection.

The obtained results (mean±standard deviation) are reported in the following Table:

| | Latency to | Duration | Rats with | Rats with | Dead | |
|---|---|---|---|---|---|---|
| Treatment | seizures | of seizures | MMS | MCC | animals | |
| groups | (sec.) | (sec) | (n/tot) | (n/tot) | <20′ | <5 h |
| 1) (n-3) | 184 ± 23 | 22 ± 9 | 1/10 | 2/10 | 1/10 | 3/10 |
| 2) (clonazepam) | 203 ± 12 | 25 ± 6 | 1/10 | 3/10 | 2/10 | 4/10 |
| 3) (n-3 + clonazepam) | 265 ± 15 | 15 ± 7 | 0/10 | 1/10 | 0/10 | 2/10 |
| 4) (control) | 12 ± 4 | 786 ± 34 | 9/10 | 1/10 | 10/10 | 0/10 |

It appears from such data that the pre-treatment with the composition of EPA and DHA ethyl esters is able to significantly protect the rats (about 90%) from major motor seizures induced by the administration of pentylenetetrazol. In the few animals not protected, the seizure is anyway delayed in the time and its duration is noticeably lower. Also a strong reduction of mortality during the convulsive period is noticed, with partial protection even during the postictal period. The effectiveness of the composition is at least similar to that of a reference drug and is noticeably potentiated if administered in combination.

### EXAMPLE 5

Two groups of 10 rats each were daily treated for 2 weeks, by i.p. route, with 50 mg/kg of a composition >80% of EPA and DHA ethyl esters, according to Example 1, composition C (group 1), and with saline solution (group 2, control). At the end all the animals were administered at 15 min. intervals a series of sub-convulsive doses of pentylenetetrazol (15 mg/kg, i.p.), so determining the number of injections required to produce an attack of clonic or tonic-clonic convulsions of forelegs and hind legs, followed by loss of straightening reflex.

Results: number of sub-convulsive doses for induction of seizure (mean±standard deviation):
16.35±3.20 (group 1, treated)
3.26±1.54 (group 2, control).

### EXAMPLE 6

Two groups of 12 mice each were treated daily for 2 weeks, by i.p. route, with 50 mg/kg of a composition >80% of EPA and DHA ethyl esters according to Example 1, composition B (group 1), and with saline solution (group 2). At the end all the animals received by i.p. route 1 mg/kg of dizocilpine, an analogue of phencyclidine able to bind the N-methyl-D-aspartate (NMDA) receptors, inducing its hypofunction and subsequent schizophreniform psychosis. The behaviour induced in the mouse consists in eliciting irregular and intense jumping (so-called popping), and its attenuation represents a valid experimental model to identify substances able to counteract the pathophysiology of schizophrenia (Deutsch S. I. et al., Neuropsychopharmacology, 15, 37, 1996; ibidem, 15, 329, 1996). The administration of dizocilpine was followed by monitoring for 30 minutes, and during the period the popping behaviour, i.e. the number of jumping of treated and control animals was registered by a suitable equipment.

Results: number of induced jumps:
45±12 (group 1, treated)
338±55 (group 2, positive control).

### EXAMPLE 7

### Materials

| | REMARKS ON FATTY ACIDS |
|---|---|
| COMPOSITION | (assays by wt %) |
| AA¹ | EPA + DHA ethyl esters = 90.6 |
| | (51.5 + 39.1, respectively) |
| BB¹ | EPA + DHA ethyl esters = 86.3 |
| | (48.2 + 38.1, respectively) |
| | (other ethyl esters of C20, C21, C22 n-3 acids = |
| | 4.6%) |
| CC¹ | EPA + DHA ethyl esters = 85.7 |
| | (46.8% + 38.9, respectively) |
| | total omega-3: 91.2 |
| FF¹ | EPA + DHA ethyl esters = 89.5 |
| | (4.3 + 85.2, respectively) |
| D3 (MaxEPA ®)² | EPA + DHA (glycerides) = 28.5 |
| | (17.1 + 11.4, respectively) |
| D6/D8³ | EPA + DHA ethyl esters = 95.3 |
| | (92.5 + 2.8, respectively) |
| | (other omega-6 = 2.6%) |

¹The compositions AA, BB, CC and FF were prepared according to the disclosure of the Example 1 of the present application as filed and fall within the definitions of the compositions A, B, C and F of said example, respectively (see the table of page 8).
²MaxEPA ® is a dietary supplement commercialised by Seven Seas Healthcare Ltd, comprising, besides to EPA and DHA, substantial amounts of saturated, monounsaturated and omega-6 polyunsaturated components; it is used in Mellor J. et al.: “Omega-3 fatty acid supplementation in schizophrenic patients”, Human Psychopharmacology, Clinical and experimental, John Wiley & Son, vol. 11, no. 1, 1996, pages 39-46-D3-(f.i., page 40, left col., “Materials”).
³EPA and DHA ethyl esters showing an assay falling within the disclosure of both U.S. Pat. No. 6,331,568-D6-[f.i. col. 3, line 28 and col. 4, letters c) and d)] and U.S. Pat. No. 6,384,077-D8-(f.i., col. 5, lines 59-60) were obtained from the distillate of the process leading to DHA ethyl ester (so-called D-1 product), according to WO8911521 (mentioned for the preparation of Composition F, example 1 of the application as filed, page 8, line 12). EPA ethyl ester from the distillate was further purified by silica gel chromatography (eluent: n-hexane) and by a final molecular distillation. Similar concentration/purification methods are summarized in U.S. Pat. No. 6,384,077-D8-(column 5, line 51 to column 6, line 3), which however does not disclose any specific experimental protocol.

Control groups were treated with olive oil, also used as a “diluent” to administer the lower doses of PUFA ethyl esters in the volume of 5-10 ml/kg in mice and 2-5 ml/kg in rats. Positive control groups were treated once with the reference substances, by ip route, diluted in saline solution.

### ADDITIONAL EXAMPLE 1

### Schizophrenia

Compositions BB and FF and MaxEPA® were screened in order to evaluate their ability to counteract the pathophysiology of schizophrenia; olive oil was used as a control.

The composition BB was also tested, following the teachings of the present application (see the specification, page 6, line 31 to page 7, line 26; specifically page 7, line 11), together with clozapine, another active principle suitable for the use of the invention in the treatment of schizophrenia.

Seven groups of 10 Swiss albino mice each were treated daily for 4 weeks, by oral route (gavage), according to the treatment scheme illustrated in the table below.

At the end of the treatment period, all the animals received by i.p. route 1 mg/Kg of dizocilpine (as described in the present specification, page 9, lines 14-18), an analogue of phencyclidine able to bind the N-methyl-D-aspartate (NMDA) receptors, inducing the hypofunction thereof and subsequent schizophreniform psychosis, resulting in irregular and intense jumping (so called “popping”). Popping attenuation represents a valid experimental model to screen substances able to counteract the pathophysiology of schizophrenia (Deutsch S. I. et al “Topiramate antagonizes MK-801 in an animal model of schizophrenia”, Eu. J. of Pharmacology 449, pp. 121-125, 2002, see page 121).

Following the administration of dizocilpine, the animals were monitored for 30 minutes and the popping behaviour, i.e. the number of jumps, was then registered.

| | | | RESULTS |
|---|---|---|---|
| GROUP | | | (No. of induced |
| No. | COMPOSITION | DOSE (mg/Kg) | jumps) |
| 1 | BB | 100 | 43 ± 15 |
| 2 | BB | 50 | 66 ± 21 |
| 3 | BB + | 25 + 2.5 | 35 ± 8 |
| | clozapine | i.p. (once) | |
| 4 | FF | 50 | 50 ± 6 |
| 5 | D3 (MaxEPA ®) | 50 | 216 ± 35 |
| 6 | D3 (MaxEPA ®) | 200 | 194 ± 27 |
| 7 | Control (olive oil) | — | 292 ± 44 |

Comments

Both compositions BB and FF were able to attenuate a schizophreniform psychosis, in a dose-related manner and in combination with a sub-effective dose of clozapine—a known drug—as well.

MaxEPA® resulted to be much less effective than compositions BB and FF.

Composition FF (comprising DHA substantially alone) resulted to be almost equipotent than composition BB whereas, according to U.S. Pat. No. 6,331,568-D6- and U.S. Pat. No. 6,384,077-D8-, EPA should be considered as the effective compound, while DHA competes with EPA and is detrimental to its activity (see, for instance, D6, col. 2, lines 30-58).

### ADDITIONAL EXAMPLE 2

### Major Depression

There are several animal models of depression as, f.i., the “chronic mild stress” CMS model and others. These models have a good predictive validity, but also some drawbacks, as they are work and time consuming (rats or mice must be exposed to the stressors for some weeks), the procedure can be difficult to establish, and so on.

The acute “Forced Swim Test” (FST), by which the animals are forced to swim in a narrow space from which there is no escape, was therefore selected so that—after an initial period of vigorous activity—they adopt a characteristic immobile posture, given up hope to escape, so called “behavioural despair”. FST is coded by Porsolt R. D. et al, “Rodent model of depression: swimming and tails suspension behavioral despair tests in rats and mice”, Curr. Protoc. Neuroscience, Chapter 8. Unit 10.A, May 2001), and is considered as a standard protocol for antidepressant drug testing.

Antidepressant drugs reduce the immobility time (provided that—as checked in complementary tests—the drug is ineffective in the usual “Spontaneous Locomotor Activity” Test).

### Experiment A

Compositions CC, FF and D6/D8 were screened in order to evaluate the antidepressant effect thereof; olive oil was used as a control.

Four groups of Swiss mice, 8 animals each group, were administered orally with 200 mg/kg/day of said compositions and control, for five weeks.

At the end of treatment, the animals underwent the Forced Swim Test (FST), a model of behavioural despair able to demonstrate the antidepressant effect of treatment.

The mice were put individually into a Plexiglas cylinder, φ10 cm, height 30 cm, containing 15 cm of water (25° C.) and left for 6 min.

The duration of immobility in the last 4 min of the test, as well as eventual swimming and climbing, were measured. The antidepressant effect results in a decrease of immobility duration. Eventual movements necessary to maintain the animal head above the water are not included in the immobility time.

### Experiment B

2 groups (1A and 1B) of Swiss mice, 8 animals each group, were treated with the composition CC, and similarly, 2 additional groups, 2A and 2B were treated with the composition FF, the dosage of both composition being the same of Experiment A: the treatment of any groups was carried out for a shorter time (2 weeks). An additional group 4A/B was kept as control.

At the end of treatment, the animals of groups 1A and 2A underwent the same FST test, while the mice belonging to groups 1B and 2B received a single injection of a known tricyclic antidepressant of the dibenzazepine group (imipramine, 10 mg/Kg, i.p. route), 30 min before FST.

Previous test had shown that imipramine at 10 mg/Kg dose was almost ineffective in FST, while 30 mg/Kg had a full effect in spite of the single dose treatment.

The results are reported in the following table.

REDUCTION OF IMMOBILITY, sec
(decrease % vs. control)

| | EXPERIMENT A | | EXPERIMENT B |
|---|---|---|---|
| GROUPS | (5 weeks) | GROUPS | (2 weeks) |
| 1 (composition CC) | 145 ± 5* | 1A | 208 ± 18 |
| | (−37.5%) | | (−9.6%) |
| | | 1B** | 134 ± 4 |
| | | | (−41.7%) |
| 2 (composition FF) | 162 ± 6* | 2A | 212 ± 13 |
| | (−30.2%) | | (−7.8%) |
| | | 2B** | 132 ± 6 |
| | | | (−42.6%) |
| 3 (D6/D8 composition) | 214 ± 16 | — | — |
| | (−7.8%) | | |
| 4 (Control) | 232 ± 4 | 4A/B | 230 ± 6 |

*the climbing time was increased to 40-50 sec. Small increases were also noted for the swimming time.
**30 min before FST, the mice were treated with 10 mg/Kg i.p. of imipramine, a known tricyclic antidepressant of the dibenzazepine group.
Note:
Blank tests proved that imipramine 10 mg/Kg i.p., if taken alone, did not reduce significantly the immobility time (218 ± 12 sec), while imipramine 30 mg/Kg i.p. was strongly effective by reducing the immobility (45 ± 8 sec) and highly increasing the climbing time (165 ± 5 sec) and partly the swimming time.

Comments

In EXPERIMENT A, the basic test of 5 weeks, the daily administration of compositions CC and FF caused a decreasing of the immobility time in the Forced Swim Test (FST) therefore showing an antidepressant action in mice. The D6/D8 composition did not result significantly effective, actually showing only a trend of effect.

In EXPERIMENT B, a shorter test of 2 weeks, also compositions CC and FF were found to be not significantly effective; they did nevertheless show a remarkable antidepressant activity when administered in combination with a single dose—ineffective if taken alone—of imipramine, as shown by the high reduction of immobility in the FST in mice.

### ADDITIONAL EXAMPLE 3

### Major Depression

Two experiments, A and B, were carried out treating orally the animal groups—for 5 and 2 weeks, respectively—with 200 mg/kg/day of the compositions CC, FF and D6/D8; olive oil was used as a control.

At the end of treatment periods, the animals underwent the Tail Suspension Test (TST), procedure equivalent to the FST, illustrated in additional example 2 above, which represents another mice model to assess the antidepressant activity of a drug (coded by Porsolt R. D. et al, supra and considered as a standard protocol for antidepressant drug testing, just as the FST).

The animals were suspended to a horizontal bar by the tail by using an adhesive tape. The experiment lasted a total of 6 minutes and during this time, the duration of immobility was measured by summing the time spent in an immobile status by each animal during the whole observation period. The immobility in the TST was simpler to distinguish from periods of movement than in the FST, where the animals have sometimes to make small movements to maintain the head above the water.

The results are reported here below:

REDUCTION OF IMMOBILITY, sec
(decrease % vs. control)

| | EXPERIMENT A | | EXPERIMENT B |
|---|---|---|---|
| GROUP No. | (5 weeks) | GROUPS | (2 weeks) |
| 1 (composition CC) | 75 ± 8 | 1A | 135 ± 12 |
| | (−49.3%) | | (−10%) |
| | | 1B* | 57 ± 6 |
| | | | (−62%) |
| 2 (composition FF) | 58 ± 5 | 2A | 122 ± 8 |
| | (−60.8%) | | (−18.7%) |
| | | 2B* | 53 ± 5 |
| | | | (−64.7%) |
| 3 (D6/D8 composition) | 120 ± 17 | — | — |
| | (−18.9%) | | |
| 4 (Control) | 148 ± 10 | 4A/B | 150 ± 7 |

*The mice were treated with the highest non-effective dose of 10 mg/Kg, i.p. of imipramine, 30 min before TST.

Comments

In EXPERIMENT A, the basic test of 5 weeks, the compositions CC and FF showed an antidepressant activity, expressed as a reduction of the immobility in the TST, much higher than that one shown by the D6/D8 composition alone.

In EXPERIMENT B, a shorter test of 2 weeks, the compositions CC and FF demonstrated a little activity, if any, but showed synergic effects by co-administration with a single, not-effective dose of imipramine.

### ADDITIONAL EXAMPLES 4-6

### Alzheimer Disease

Cholinergic dysfunction and abnormal protein deposition in the brain (amyloid-β protein, Aβ, released from its amyloid precursor protein—APP— by secretase enzymes), leading to memory and learning impairment, are typical of Alzheimer Disease (Goodman & Gilman's, The Pharmacological Basis of Therapeutics, 10^{th} ed., McGraw-Hill, 2001, pp. 560-562).

### ADDITIONAL EXAMPLE 4

### Alzheimer Disease

Cholinesterase inhibitors and, in particular the acetylcholinesterase (AChE) ones, represent most of the current therapeutic drugs able, through a symptomatic mechanism of action, to elevate brain acetylcholine levels, thus improving learning and memory.

Rat AChE and butyrylcholinesterase (BChE) were prepared according to literature [Yu Q. S. et al, “Total Syntheses and Anticholinesterase Activities of (3aS)—N(8)-Norphysostigmine, (3aS)—N(8)-Norphenserine, Their Antipodal Isomers, and Other N(8)-Substituted Analogues”, J. Med Chem, 40, pp. 2895-2901, 1997], and the compositions CC, FF and D6/D8 were tested to determine the concentration thereof suitable to inhibit 50% enzyme activity (IC 50).

In this in vitro test, the ethyl esters were hydrolyzed to free acid by standard procedures before incubation and tacrine, an AChE inhibitor, was used as an external reference standard.

Acetyl-methylthiocholine and butyrylthiocholine were used as substrates for the two enzymes, and incubation was performed in the presence of 5,5′-dithiobis-(2-nitrobenzoic acid), by measuring the released yellow thionitrobenzoate anion by spectrophotometry at 412 nm.

The analysis of the enzyme inhibitory activity was carried out at 37° C. and in a pH=8, 0.1 M phosphate buffer, calculating the IC 50 by interpolation on log scale, after subtracting the non-specific background levels.

The results are reported here below, as a mean of 5 tests.

Results

| COMPOSITION | AChE (IC 50) - μM | BChE (IC 50) - μM |
|---|---|---|
| CC | 1.25 ± 0.08 | 85 ± 7 |
| FF | 0.42 ± 0.06 | 27 ± 3 |
| D6/D8 | 4.18 ± 0.12 | 284 ± 18 |
| Tacrine (control) | 28 ± 5** | 1.5 ± 0.2 |

**Expressed as nM

Comments

Generally, the tested omega-3 compositions were much less effective as cholinesterase inhibitors than tacrine; both compositions CC and FF showed an activity higher than the one measured for the prior art composition. Composition FF resulted to be about 10 times more effective than the prior art one.

While less effective than selective cholinesterase inhibitors, omega-3 based compositions are however known to be much better tolerated in vivo and free from cholinergic effects (such as endogenous secretion decrease) in comparison with tacrine, a selective cholinesterase inhibitor.

### ADDITIONAL EXAMPLE 5

### Alzheimer Disease

The protection against the impairment of memory and of learning ability in rats infused with amyloid beta Aβ peptide (1-40) into the cerebral ventricle (icv), by pre-treatment with the compositions CC, FF and D6/D8 had been measured in the present test.

The surgical techniques for preparing Aβ-infused rats were essentially those reported by Yamada K. et al, “Protective effects of idebenone and alfa-tocopherol on beta-amyloid-(1-42)-induced learning and memory deficits in rats: implication of oxidative stress in beta-amyloid-induced neurotoxicity in vivo”, Eu. J. of Neuroscience, vol. 11, pp. 83-90, 1999—see page 83). Each rat was anesthetized with pentobarbital (50 mg/Kg, i.p.), the skull was exposed, a hole was drilled and a suitable catheter connected to a mini-osmotic infusion pump was inserted into the left ventricle.

Preliminarily tests clearly indicated that the infusion of Aβ peptide into the rat cerebral ventricle impaired memory and learning ability in rats.

The memory impairment was assessed using an 8-arm radial maze, training the rats to acquire a reward (food pellet) at the end of each of 4 arms.

The performance involved the “reference memory” (RM) that is “the information that should be retained until next trial”, whose error (RME) is represented by the entries into unbaited arms.

The error of the “working memory”, repeated entries into arms already visited, was less significant.

In all, 4 groups of Wistar rats, 10 rats each, were treated for 4 weeks: all groups 1 to 4 were infused by icy route with Aβ peptide (Sigma Co), while group 2 and 3 also received orally compositions CC and FF and group 4 the D6/D8 composition, all of them at 200 mg/Kg dose.

Results

The results for the last 3 daily blocks of trials, each block being constituted by 6 trials, are expressed in the order as the mean number of RMEs for each group, as reported here below.

| | Number of RMEs (mean ± SEM*) - | | |
|---|---|---|---|
| GROUP No. | last 3 blocks of trials- | | |
| 1 (Aβ infusion) | 2.6 ± 0.3 | 2.6 ± 0.1 | 2.5 ± 0.2 |
| 2 (Composition CC) | 1.6 ± 0.1 | 1.6 ± 0.2 | 1.4 ± 0.2 |
| 3 (Composition Ff) | 1.6 ± 0.3 | 1.3 ± 0.2 | 1.5 ± 0.1 |
| 4 (D6/D8 Composition) | 2.0 ± 0.1 | 2.3 ± 0.2 | 2.1 ± 0.1 |

*SEM: Standard Error Mean

Comments

The results demonstrated that, while the infusion of peptide Aβ (1-40) in the rat cerebral ventricle impaired reference memory, the oral co-administration of the test substances was able to lower the RME score, in spite of the continuous infusion of Aβ into the brain. Lower error values were obtained with the compositions CC and FF, which were similar each other, than with the D6/D8 composition.

### ADDITIONAL EXAMPLE 6

### Alzheimer Disease

The trial was addressed to control the effect of the compositions AA, FF and D6/D8 composition on the brain total amyloid burden, when administered to a transgenic mouse model of Alzheimer disease.

Tg2576 (Hsiao K. et al, “Correlative memory Deficits, AP 3 Elevation, and Amyloid Plaques in Transgenic Mice”, Science, Vol. 274, pp. 99-102, 1996) is a transgenic mouse overexpressing the human Alzheimer β-amyloid (Aβ) precursor protein which leads to increase in Aβ (1-40) and Aβ (1-42) and impairment in normal learning and memory by 9-10 months of age.

Four groups of five Tg 2576 mice each (weight 365±21 g, age 16 months), were treated for 6 weeks by oral route with 200 mg/Kg of compositions AA (group 1), FF (group 2), and D6/D8 (group 3); olive oil (group 4) was the control.

At the time of sacrifice, the animals were perfused before brain dissection with saline and HEPES buffer (4-2-hydroxyethyl-1-piperazineethanesulfonic acid, pH=7.2), containing a cocktail of protease inhibitors, by proceeding as described in Lim G. P. et al, “Ibuprofen Suppresses Plaque Pathology and Inflammation in a Mouse Model for Alzheimer's Disease”, J of Neuroscience vol 20 (15), pp. 5709-5714, 2000.

The brain cortex region was dissected and tissue was homogenized in 10 vol of TBS (TRIS—tris-hydroxymethyl-methylamine—Buffered Synthetic medium, pH=8.0) containing protease inhibitors as above.

The samples were sonicated briefly and centrifuged for 20 min at 4° C. and the soluble fraction was used for dosing Aβ by ELISA. The insoluble pellet was then solubilized and sonicated in 70% formic acid, centrifuged and the extract was neutralized with 0.25 M TRIS buffer (pH=8.0), containing 30% acetonitrile and 5M sodium hydroxide.

The soluble and solubilized Aβ fractions were then loaded onto the wells of ELISA (Enzyme-Linked Immunosorbent Assay) plates for analysis.

A sandwich ELISA method for the detection of total Aβ, as disclosed in Lim G. P. et al, supra, was used according to known methods.

Fractions Aβ40 and Aβ42 were not dosed. Monoclonal 4G8 against Aβ17-24 (Senentek, Calif.) was used as the capture antibody at 3 μg/ml in 0.1 M carbonate buffer pH 9.6. Blocking was completed with 2% bovine serum albumin in TBS medium.

Processed samples were diluted with EC buffer (TBS containing 0.1 mM EDTA, 1% bovine serum albumin BSA, and 0.05% 3-(3-cholamidopropyl-dimethylamonium)-1-propanesulfonic acid CHAPS, pH=7.4) containing protease inhibitors, and soon after, equal volumes of each sample, and then of the detector antibody 10 G4 against Aβ5-13, conjugated to horseradish peroxidase, were loaded onto the wells and left overnight at 4° C.

After treatment with the Microwell peroxidase substrate kit, the OD450 (optical density at 450 nm) values were read with a microplate spectrophotometer and Aβ concentrations are interpolated from the OD450 values of a standard curve obtained with known Aβ amounts.

Results

| | SOLUBLE Aβ | INSOLUBLE Aβ |
|---|---|---|
| | pg/μg protein | ng/pellet |
| GROUPS | (% decrease vs. control) | (% decrease vs. control) |
| 1 (composition AA) | 10.1 ± 2.3 (−44.8%) | 9.8 ± 0.5 (−70.0%) |
| 2 (composition FF) | 9.5 ± 1.4 (−48.1%) | 10.3 ± 1.2 (−68.5%) |
| 3 (D6/D8 | 14.6 ± 3.5 (−20.2%) | 22.6 ± 1.0 (−30.9%) |
| composition) | | |
| 4 (control) | 18.3 ± 5.2 | 32.7 ± 2.2 |

Comments

All the compositions under test were able to decrease the presence of amyloid-beta protein in the brain of transgenic mice tested as model for Alzheimer disease.

The compositions AA and FF administered to groups 1 and 2, were much more potent than that of the prior art (group 3).

In detail, a high activity was shown against soluble amyloid-β, a potential toxic form of amyloid, and particularly against insoluble amyloid-β, which is a constituent of the brain plaques in the AD-disease.

### EXAMPLE 8

This experiment shows the use and the effects of the claimed compositions in the manic-depressive syndrome.

The used compositions were CC and FF and a comparison substance was D3 (MaxEpa, Trade Mark), all compositions as detailed in the “ADDITIONAL EXAMPLES” described above in Example 7. Furthermore, another new comparison substance, coded D19/D20, was also used, having 44.2% b.w. of EPA and 23.4% b.w. of DHA (ethyl esters): it was thought that this composition could properly reproduce the compositions used in the prior arts D19 and D20. It has been easily prepared according to a known procedure already adopted for the preparation of other compositions of EP 1706106 (see Example 1), that is by two-step molecular distillation as reported by U.S. Pat. No. 5,130,061. Olive oil was also used for the control groups. This experiment is referred to below as “ADDITIONAL EXAMPLE 7”.

Based on the above results and on the availability of the D19/D20 reference, the main test (5 week treatment) of the ADDITIONAL EXAMPLE 3 described above in Example 7 was repeated to check the effects of two claimed compositions, namely compositions BB and FF, in the major depression syndrome, by using MaxEpa and the above described D19/D20 as comparison substances. Olive oil was used as a control. This experiment is referred to below as “ADDITIONAL EXAMPLE 8”.

### ADDITIONAL EXAMPLE 7

### Bipolar Disorder, Also Referred to Herein as Manic Depressive Syndrome

Bipolar disorders typically alternate episodes of depression and episodes of mania. The depressive phase consists of anhedonia, feelings of guilt, and so on, reaching thoughts of death or suicide, and is generally indistinguishable from that of unipolar depression. The manic phase is characterized by a heightened mood, inflated self esteem, increased physical energy and mental activity, irritability, etc., sometimes including psychotic symptoms as hallucinations and delusions. Hypomania is a less severe form of mania. Modeling bipolar disorders in animal is very difficult as the disease is cyclic, has high heterogeneity of effects, and limited knowledge of the underlying pathophysiology.

Therefore models of the depressive phase make use of models validated in the context of depression research, while symptoms of mania are separately modeled in animals, such as f.i. increased activity, reduced need for sleep, and aggressive and risk-taking behavior. The present experiment is designed to study the effect of omega-3 compositions in an amphetamine-induced behavioral model of mania in rats.

Compositions CC and FF, containing about 85% by weight of EPA+DHA and more than 90% b.w. of total omega-3 compounds, and respectively about 90% b.w. of DHA, all as ethyl esters, were administered at the fixed dose of 100 mg/kg, while MaxEpa—a composition of glycerides of EPA+DHA at about 30%—was administered at 100 and 300 mg/kg. Due to its content of 67-68% EPA+DHA, D19/D20 was administered at the dose of 150 mg/kg.

Composition CC was also treated in combination with 20 mg/kg sodium valproate, a well known anti manic agent however in a dose proven to be sub-effective in preliminary experiment, and 2 further groups of rats were treated with olive oil and kept as negative and positive control groups.

In all, 8 groups of male Wistar rats, 10 animals each, were used and treated daily for 3 weeks by oral route with the oily compositions (gavage), according to the following administration scheme. Valproate was injected by i.p. route for the last 3 days.

At the end of the treatment period, all the animals were treated once (acute test) with 1.0 mg/kg amphetamine sulfate dissolved in saline by i.p. route (with the only exclusion of the “negative” control group which was treated with saline), by inducing hyperactivity and manic symptoms (Antoniou K. et el, Neurosci Biobehav Rev 1998; 23,189).

The animals were soon after submitted to testing for locomotor and exploratory behavior, by using a large open field constituted by a 120×120 cm transparent Plexiglas platform, without walls, 100 cm above the floor. A center square of 40×40 cm was defined as the “Center area” of the open field, to check the exploratory activity of the animals (Decker S. et al, Prog Neuropsychopharmacol Biol Psychiatry 2000; 24, 455). Data were collected by a video tracking system suitable to study the spatial behavior and monitored for 40 minutes.

The results are collected in the Table here below as mean values +/−S.D.:

| Group | | | Distance | Visits to |
|---|---|---|---|---|
| No. | Composition | Dose (mg/kg) | Traveled (cm) | center (No.) |
| 1 | CC | 100 | 78 +/− 15 * | 20 +/− 5 * |
| 2 | CC + valproate | 100 + 20 | 61 +/− 4 * | 15 +/− 2 * |
| | | i.p. × 3 days | | |
| 3 | FF | 100 | 71 +/− 7 * | 18 +/− 4 * |
| 4 | MaxEpa | 100 | 196 +/− 22° | 40 +/− 7° |
| 5 | MaxEpa | 300 | 188 +/− 23° | 38 +/− 13° |
| 6 | D19/D20 | 150 | 166 +/− 17 *° | 35 +/− 14° |
| 7 | Positive control | 100 (olive oil) | 214 +/− 18° | 46 +/− 3° |
| 8 | Negative | 100 (olive oil) | 63 +/− 8 * | 14 +/− 5 * |
| | control | | | |

Doses administered orally for 3 weeks.
Groups 1-7 treated with amphetamine, 1.0 mg/kg. i.p., once.
°P < 0.01 vs group 7 (positive control)
°P < 0.01 vs group 8 (negative control)

Conclusions

By repeated administration, compositions CC and FF were able to reduce the hyperlocomotion induced by an acute administration of amphetamine in the open field test in rats (see the distance traveled by the animals of groups 1 and 3 in comparison to that of group 7, positive control).

The repeated administrations were thought to be necessary in this animal model, to slowly saturate the endogenous pool of lipids and phospholipids, also taking into account the short term effect of amphetamine.

The reduction of rat hyperactivity did not affect the levels of spontaneous locomotor activity, as shown by comparison with the behavior of the negative control group untreated with amphetamine (group 8).

A combination with a well known anti-manic agent, as sodium valproate, given for short times and in low doses (proven to be ineffective when taken alone) showed a synergic effect (group 2).

The low concentration composition of EPA and DHA (MaxEpa) was not effective, not even given in doses high enough to compensate its low concentration. It is thought that other components of the composition may have some opposite effect (groups 4 and 5).

The intermediate EPA and DHA composition (D19/D20), in a dose suitable to reach the same amount of EPA and DHA as with compositions CC and FF, showed a significant, but very limited reduction of the induced hyperlocomotion, still remaining much higher than that of amphetamine-untreated, negative controls (group 8).

Similar effects were obtained by monitoring the number of visits to the center of the open field, which represents a risk-taking behavior, with normalization of the amphetamine-induced increase of visits for the same groups as above. Composition D19/D20 had however a very low activity, if any, and its effect did not reach the threshold of statistical significance toward the positive group (group 7).

### ADDITIONAL EXAMPLE 8

### Major Depression

The principles of the test (Tail Suspension Test, TST), which is considered as a standard protocol for testing antidepressant drugs, were explained in the previous “Additional Example 3” above in Example 7.

Shortly, after drug treatment the experimental animals were suspended to a horizontal bar by the tail, by using an adhesive tape. During the following 6 minutes, the duration of immobility was measured by summing the time spent in an immobile status by each animal during the whole observation period. Immobility represents giving up hope to escape, so called “behavioural despair”, and immobility time may be shortened by antidepressant drugs.

In comparison with to the previous “Additional Example 3”, only the main test with the longer administration time of 5 weeks was carried out. Five groups of 10 male Swiss mice each, were used in all.

Compositions BB and FF, containing more than 85% by weight of EPA+DHA and about 90% b.w. of total omega-3 compounds, and respectively about 90% b.w. of DHA, all as ethyl esters, were given by oral route at the fixed dose of 200 mg/kg. The reference composition MaxEpa, containing a total of about 30% of EPA and DHA, was administered at a 600 mg/kg dose, while D19/D20(EPA and DHA, ratio about 2:1, content 67.6%) was given at the dose of 300 mg/kg. Oral olive oil was administered as a negative control.

At the end of the treatment period, all the animals underwent the TST test, by measuring the total immobility time during the 6 minute controls.

The results are reported in the Table here below (means+/−S.D.):

| | | | Reduction of immobility | |
|---|---|---|---|---|
| Group | | Dose | Seconds | Decrease vs control |
| No. | Composition | (mg/kg) | (Means +/− S.D.) | (%) |
| 1 | BB | 200 | 68 +/− 12* | −53.4 |
| 2 | FF | 200 | 65 +/− 9* | −55.5 |
| 3 | MaxEpa | 600 | 132 +/− 15 | −9.6 |
| 4 | D19/D20 | 300 | 124 +/− 21 | −15.1 |
| 5 | Olive oil | 300 | 146 +/− 18 | — |
| | (Control) | | | |

Doses administered orally for 5 weeks.
*P < 0.01 vs control group.

Conclusions

After long term administration, compositions BB and FF were able to significantly decrease the immobility period of mice submitted to the Tail Suspension Test in comparison with untreated animals, demonstrating therefore a clear antidepressant activity.

The MaxEpa composition, containing a much lower concentration of omega-3 components, showed a very low activity, if any, in spite it was administered at a dose much higher, sufficient to give similar amounts of EPA and DHA.

D19/D20 showed a limited activity in decreasing the immobility time compared with the untreated animals, however the induced reduction remained very far from that induced by the concentrated compositions.

### EXAMPLE 9

Described herein is the composition b) comprising docosahexaenoic acid (DHA, C22:6n-3) and/or the pharmaceutically acceptable derivates and/or precursors thereof, in a concentration not lower than 70% by weight of the total fatty acid weight in the composition, for the preparation . . . (Etc.). Successively, the composition description was limited according to composition F of the reported Example 1 of the Application, to claim—as composition d)—“DHA ethyl ester >80 (%, by weight) and EPA ethyl ester <15, wherein EPA+DHA ethyl esters >85, for the preparation . . . (etc.).

In the ADDITIONAL EXAMPLES, presented in Example 7 above, to better describe the invention, the selected composition FF, comprising DHA ethyl ester 85.2% b.w., EPA ethyl ester 4.3% b.w. and then DHA+EPA ethyl esters 89.5% b.w., was experimentally used.

In the present Example, a few experiments described above in the ADDITIONAL EXAMPLES (described above in Example 7) were repeated by using—instead of composition FF— the new composition FFF, wherein the % content of EPA ethyl ester was reduced to “zero”, that is below the detectable limit under the gas-chromatographic GC analysis (see EU Pharmacopoeia, Suppl. 2000, monograph 1250), which was in the experimental conditions lower than 0.001% (10 p.p.m.).

To this purpose, the previous sample FF was submitted to 3 consecutive 2-step molecular distillations, in each of them discarding the lower boiling fraction richer in EPA (about 30% of the feed). The final high boiling fraction was further purified by column chromatography on silica gel showing by GC analysis to contain DHA 98.2% b.w. and EPA 0% (composition FFF). The principles of preparation and purification methods are those reported in the literature of Example 1 of the Description.

### ADDITIONAL EXAMPLE 1

### Bis(Schizophrenia)

Composition FFF was used to evaluate its ability in counteracting the pathophysiology of Schizophrenia in mice and compared with a control group treated with olive oil. In all, two groups of 10 Swiss albino male mice were treated daily with 50 mg/kg of the substances, for four weeks.

The details of the method are as reported in “ADDITIONAL EXAMPLE 1” described above in Example 1.

At the end of the treatment period and after administration of dizocilpine, the popping behavior was monitored for 30 minutes, with the following results:

| | | Dose | Results (N. of | % Reduction of |
|---|---|---|---|---|
| Group n. | Composition | (mg/kg) | induced jumps) | jumps |
| 1 | FFF | 50 | 46 +/− 7 | 83.9 |
| 2 | Control | 50 | 285 +/− 32 | |

Comments:

Composition FFF, containing DHA ethyl ester 98.2% b.w. and free from EPA, was quite effective in a Schizofrenia animal model and was almost equipotent in comparison with composition FF used in a previous experiment and containing a limited amount of EPA ethyl ester (4.3% b.w.).

### ADDITIONAL EXAMPLE 2

### Bis(Major Depression)

Composition FFF was used in an animal model of depression, in comparison with a control group treated with olive oil. Two groups of 10 Swiss albino male mice were treated daily with 200 mg/kg of the 2 substances for 5 weeks by oral route.

The adopted method was the FST, according to which the animals, after an initial period of vigorous swimming, adopted an immobile posture, given up hope to escape. As shown in more detail in Additional Example 2, Experiment A, the decrease of immobility time (seconds) represent the effect of the antidepressive drug.

Results were as follows:

| Group | | Dose | Results (Reduction | % Decrease of |
|---|---|---|---|---|
| n. | Composition | (mg/kg) | of immobility- sec-) | immobility |
| 1 | FF | 200 | 160 +/− 10 | 34.7 |
| 2 | Control | 200 | 245 +/− 12 | |

Comments:

Composition FFF, containing DHA ethyl ester 98.2% b.w. and free from EPA, was quite effective as an antidepressive drug in the FST animal model, and it has similar potency in comparison with composition FF used in the previous experiment.

### ADDITIONAL EXAMPLE 7

### Bis(Bipolar Disorder, Also Referred to Herein as Manic Depressive Syndrome)

Composition FFF was evaluated for its activity in an amphetamine-induced behavioral model of mania in rats, resulting in increased activity and risk-taking behavior of the animals.

The composition was given orally at the daily dose of 100 mg/kg for 3 weeks to a group of 10 male Wistar rats weighing 230+/−12 g, in comparison with a control group of 10 animals treated with olive oil.

At the end of treatments, all the rats were given 1.0 mg/kg of amphetamine sulphate by i.p. route, by inducing hyperactivity and manic symptoms, and tested for locomotor and exploratory behavior in the “open field” test, as explained in more detail in the previous ADDITIONAL EXAMPLE 7 described above in Example 8.

The distance traveled and the visit to the field center were monitored and the results are reported in the Table here below:

| | | Dose | Distance | Visit to the |
|---|---|---|---|---|
| Group n. | Composition | (mg/kg) | traveled (cm) | center (N.) |
| 1 | FF | 100 | 74 +/− 8 | 15 +/− 6 |
| 2 | Control | 100 | 210 +/− 21 | 47 +/− 10 |

Comments:

Composition FFF, containing DHA ethyl ester 98.2% b.w. and free from EPA was able to significantly reduce the hyperlocomotion induced by a single amphetamine administration in the “open field” test in the rat.

A similar reduction was obtained for the visits to the open field center, which represent a risk-taking behavior. The effects obtained with composition FFF were quite similar to those of composition FF used in a previous experiment.

What is claimed is:

1. A method for the treatment or prevention of at least one of manic-depressive syndrome and major depression, the method comprising administering to a subject a composition consisting essentially of, in a concentration expressed as % by weight of the total fatty acid weight in the composition, DHA ethyl ester >80 and EPA ethyl ester <15, wherein EPA+DHA ethyl esters >85.

2. The method of claim 1, wherein the subject suffers from manic-depressive syndrome.

3. The method of claim 1, wherein the subject suffers from major depression.

4. The method of claim 1, wherein the subject suffers from manic-depressive syndrome and major depression.

5. The method of claim 1, wherein the composition is administered by oral route.

6. The method of claim 1, wherein the composition is in the form of soft gelatine capsules.

7. The method of claim 1, wherein the composition is administered at the dose of 0.1-5 g/day.

8. The method of claim 1, wherein the composition is administered at the dose of 1-2 g/day.

9. The method of claim 1, wherein the composition is administered separately, as a coadjuvant or an auxiliary drug, from at least another drug effective for the treatment of at least one of manic-depressive syndrome and major depression.

10. A method for the treatment or prevention of manic-depressive syndrome comprising administering to a subject a composition consisting essentially of, in a concentration expressed as % by weight of the total fatty acid weight in the composition, DHA ethyl ester >34 and EPA ethyl ester >40, wherein EPA+DHA ethyl esters >80, the total ethyl esters of n-3 acids being >90.

11. The method of claim 10, wherein the composition is administered by oral route.

12. The method of claim 10, wherein the composition is in the form of soft gelatine capsules.

13. The method of claim 10, wherein the composition is administered at the dose of 0.1-5 g/day.

14. The method of claim 10, wherein the composition is administered at the dose of 1-2 g/day.

15. The method of claim 10, wherein the composition is administered separately, as a coadjuvant or an auxiliary drug, from at least another drug effective for the treatment of manic-depressive syndrome.

16. A method for treating or preventing at least one disease selected from the group consisting of: major depression and manic-depressive illness in a subject, the method comprising administering to the subject a therapeutically effective dose of a product for oral ingestion comprising a composition consisting essentially of, in a concentration expressed as % by weight of the total fatty acid weight in the composition, DHA ethyl ester >80 and EPA ethyl ester <15, wherein EPA+DHA ethyl esters >85.

17. The method of claim 16, wherein the composition is present in the range of about 0.05 gram to about 5 gram per dose of the product for oral ingestion.

18. The method of claim 17, wherein the composition is present in the range of about 0.1 gram to about 2.0 gram per dose of the product for oral ingestion.

19. A method for treating or preventing manic-depressive syndrome in a subject, the method comprising administering to the subject a therapeutically effective dose of a product for oral ingestion comprising a composition consisting essentially of, in a concentration expressed as % by weight of the total fatty acid weight in the composition, DHA ethyl ester >34 and EPA ethyl ester >40, wherein EPA+DHA ethyl esters >80, the total ethyl esters of n-3 acids being >90.

20. The method of claim 19, wherein the composition is present in the range of about 0.05 gram to about 5 gram per dose of the product for oral ingestion.

21. The method of claim 20, wherein the composition is present in the range of about 0.1 gram to about 2.0 gram per dose of the product for oral ingestion.

22. A product for oral ingestion for the treatment or prevention of at least one of manic-depressive syndrome and major depression in a subject comprising a composition consisting essentially of, in a concentration expressed as % by weight of the total fatty acid weight in the composition, DHA ethyl ester >80 and EPA ethyl ester <15, wherein EPA+DHA ethyl esters >85.

23. A product for oral ingestion for the treatment or prevention of manic-depressive syndrome in a subject comprising a composition consisting essentially of, in a concentration expressed as % by weight of the total fatty acid weight in the composition, DHA ethyl ester >34 and EPA ethyl ester >40, wherein EPA+DHA ethyl esters >80, the total ethyl esters of n-3 acids being >90.
